(19) United States
(12) Patent Application Publication
(10) Pub. No.: US 2008/0087568 A1
(43) Pub. Date: Apr. 17, 2008

(54) Toothbrush kit with releasable handle and disposable pre-pasted brush heads

(76) Inventor: S. Jabri, Burr Ridge, IL (US)

Correspondence Address: MILLER, MATTHIAS & HULL, ONE NORTH FRANKLIN STREET, SUITE 2350, CHICAGO, IL, 60606, US

(21) Appl. No.: 11/548,834

(22) Filed: Oct. 12, 2006

(51) Int. Cl. B65D 69/00; A46B 11/00; A46B 7/04; A61B 19/02

(52) U.S. Cl. 206/570; 15/104.94; 15/176.1; 206/63.5

(57) ABSTRACT

A dental hygiene kit is disclosed which is particularly useful for small children and traveling adults, and which includes a package having one large initially-sealed compartment for storing a handle and a plurality of smaller, initially-sealed compartments for storing disposable pre-pasted brush heads. The compartments are sterile in the initially seated condition. The handle comprises a fork and with two spaced-apart shaped prongs. Each brush head comprises a plurality of bristles connected to a body. The bristles are coated with toothpaste thereby avoiding wasted toothpaste and mess. Each brush head body comprises a shaped hole with sidewalls for frictionally and releasably receiving the prongs of the handle. After each use, the brush heads are discarded. The disclosed kit helps children learn good brushing habits while avoiding wasted and swallowed toothpaste.

### BACKGROUND

1. Technical Field

This disclosure relates generally to toothbrushes and oral hygiene, and more particularly, to a disposable, single-use pie-pasted brush heads for use with a reusable handle. A plurality of pie-pasted heads are packaged with one handle in the form of a kit, that is particularly useful in teaching young children good oral hygiene habits without wasting undue amounts of toothpaste. The disclosed kits also make excellent travel accessories

2. Description of the Related Art

Disposable toiletry items offer great convenience to travelers and others needing to carry their personal necessities. For example, many motels, hotels and health clubs commonly provide disposable, single-use supplies of shampoo, soap, hand lotion or other such items, for the convenience of their guests or members.

Although the toiletry articles listed above have long been available in the art, toothpaste and toothbrushes have heretofore presented certain obstacles to being provided in single-use, disposable form. Even though they are intended to be used together, toothbrushes and toothpaste are ordinarily supplied as separate articles, which means a doubling of packaging and handling costs, which runs counter to the convenience and low cost that ale essential to a single-use, disposable product. Furthermore, only a small amount of toothpaste is required for personal, one time use, to the point where providing individual packaging for such small amounts is not economical, and the packaging is so small that it becomes difficult and inconvenient to open and dispense onto the toothbrush.

A variety of devices have been proposed for providing a toothbrush with an integral supply of toothpaste Examples of disposable toothbrushes with integral reservoirs of toothpaste include U.S. Pat. Nos. 4,522,524, 4,844,641 and Published U.S. Patent Application Nos. 2005/0284499, 2006/0115317, and 2004/0022576

In most instances, the above disclosures include a reservoir for the toothpaste that is located in handle of the structure, usually so that the toothpaste is dispensed as the handle is squeezed by the user. In general, such prior devices are too complex and costly to be manufactured for one-time, disposable use. Likewise, the configuration of most of the prior devices requires them to be filled or refilled by the user, and they are not suitable for being filled with a small amount of toothpaste in a high-speed manufacturing process. Moreover, most of the prior devices employ an elongate passage or conduit to convey the toothpaste from the handle to the brush head of the assembly, which is not only difficult to manufacture economically, but also tends to lead to hardening and clogging of the toothpaste if the assembly is left in storage for an extended period, as can be expected with a disposable product. Further, such complex designs require dexterity that is beyond the ability of young children.

Further, one problem not addressed by the above devices or the prior art in general is toothpaste waste, which is common among young children. Specifically, children tend to use too much toothpaste and often, excess toothpaste is splattered on the sink or bathroom counter much to the chagrin of parents. Also, and perhaps more important, when children use too much toothpaste, they tend to swallow it, unnecessarily ingesting fluoride and saccharin and toothpaste ingredients.

Therefore, there is a need for an improved toothbrush for young children and for adults which provide pre-pasted toothbrushes and that is economical and practical.

### SUMMARY OF THE DISCLOSURE

In satisfaction of the aforenoted needs, a dental care kit is disclosed which comprises a handle and a plurality of disposable pie-pasted brush heads. The handle comprises an end comprising a shaped prong. Each disposable pie-pasted head comprises a body connected to a plurality of bristles. At least some of the bristles are coated with toothpaste. Each body of each head further comprises a shaped opening for frictionally and releasably receiving the shaped prong of the handle.

In a refinement, the end of the handle is flexible thereby permitting the prong to be moved into and out of frictional engagement with one of the shaped openings of one of the heads.

In a refinement, the end of the handle comprises a pair of spaced-apart shaped prongs that can be frictionally and releasably received in one of the shaped openings of one of the heads.

In a refinement, the spaced-apart shaped prongs may be squeezed together to release the prongs from one of the shaped openings.

In a refinement, the shaped prong or prongs comprise at least one barb and each shaped opening comprises a sidewall with at least one ridge for frictionally engaging the barb of the prong. In a related refinement, the shaped prong or prongs comprise a plurality barbs and each shaped opening comprises a sidewall with ridges for frictionally engaging the barbs of the prong.

In a refinement each prong is connected to the end of the handle by a rounded transition section. The rounded transition sections extend away from each other and comprise an outer surface that serves as a grip to facilitate the pressing of the prongs together.

In a refinement, the kit further comprises packaging wherein the packaging comprises a plurality of connected and sealed compartments with the handle being disposed within one of the sealed compartments and each head is within one of the other sealed compartments. In other words, the handle is disposed within its own sealed compartment and each pre-pasted brush head is disposed within its own sealed compartment.

In a refinement, the compartments are sterile.

In a refinement, the kit comprises one handle and from about six to about 30 pre-pasted brush heads.

In a refinement, the kit comprises one handle and from about 10 to about 24 pre-pasted brush heads.

In a refinement, a dental hygiene kit is disclosed which comprises a packaging comprising a clear polymeric material and one large initially-sealed compartment for storing a handle and a plurality of smaller, initially-sealed compartments for storing disposable pre-pasted brush heads. The compartments are connected to at least two other compartments and each compartment is sterile when sealed. The handle comprises a fork and with two spaced-apart shaped prongs. Each head comprises a plurality of bristles connected to a body; at least some of the bristles are coated with toothpaste. Each body comprises a shaped hole with sidewalls for frictionally receiving the prongs of the handle.

A toothbrush with a pre-pasted removable brush head is disclosed. The disclosed toothbrush comprises a handle comprising an elongated grip that terminates at a forked end with a pair of spaced-apart the barbed prongs. The prongs are connected to the grip by a flexible transition section that, when squeezed, causes the prongs to move inwardly towards each other. The toothbrush further comprises a head comprising a body connected to a plurality of bristles. At least some of the bristles are pre-coated with toothpaste. The head body comprises a shaped opening comprising sidewalls with ridges for frictionally engaging the barbed prongs.

In a refinement, each prong is connected to an end of the handle by a rounded transition section. The rounded transition sections extend away from each other and comprise an outer surface that serves as a grip to facilitate the pressing of the transition sections and prongs together to release the prongs from the shaped opening of the head.

Other advantages and features will be apparent from the following detailed description when read in conjunction with the attached drawings

### BRIEF DESCRIPTION OF THE DRAWINGS

For a more complete understanding of the disclosed methods and apparatuses, reference should be made to the embodiment illustrated in greater detail on the accompanying drawings, wherein:

FIG. 1 is a perspective view of a kit made in accordance with this disclosure with 20 pre-pasted brush heads and a handle;

FIG. 2 is an exploded view illustrating the handle removed from the kit in preparation for insertion of the handle and to one of the pre-pasted brush heads;

FIG. 3 is a partial perspective view of the handle prior to insertion in one of the pre-pasted blush heads;

FIG. 4 is a top perspective view of the handle as inserted into a pre-pasted blush head;

FIG. 5 is a bottom perspective view of the handle and pre-pasted brush head combination shown in FIG. 4;

FIG. 6 is an enlarged partial sectional and perspective view of the handle and a pie-tasted brush head illustrating the detachable connection between the handle and brush head;

FIG. 7 is another enlarged partial sectional and perspective view of the handle and the pre-tasted brush head illustrated in FIG. 6; and

FIG. 8 is a perspective view of yet another kit made in accordance with this disclosure particularly illustrating the heads inverted with in their respective compartments so the handle may be connected to a head through a top frangible cover sheet.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION OF THE PRESENTLY PREFERRED EMBODIMENTS

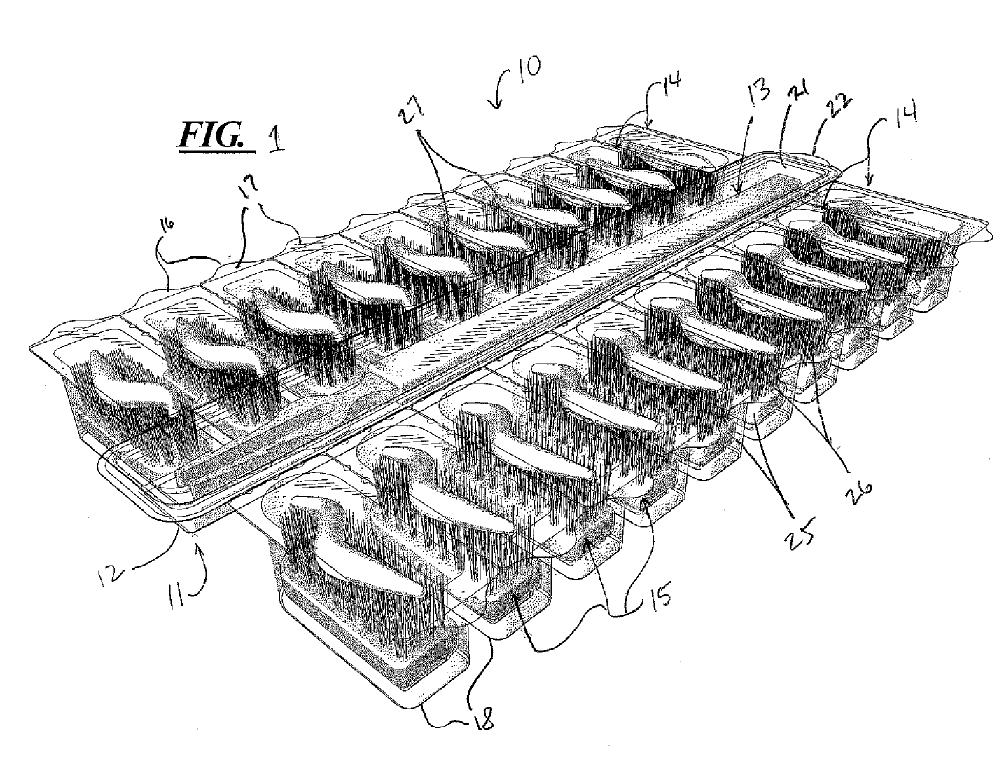

Turning first to FIG. 1, a kit 10 made in accordance with this disclosure is shown and described. The kit 10 includes a multi-faceted packaging 11 that, in the embodiment shown in FIG. 1 includes a central trough portion 12 that accommodates a reusable handle 13 and a plurality of the side compartments, shown generally at 14 that accommodate pre-pasted brush heads, shown generally at 15. In the embodiment 10 shown in FIG. 1, the reusable handle 13 is disposed centrally between two tows of brush heads 15. Each compartment 14 that accommodates a brush head 15 includes a removable plastic cover 16, equipped with a pull tab 17. Each compartment 14 also includes a lower trough or storage area 18 for accommodating a single blush head 15. When sealed with the cover or membrane 16, each compartment 14 is sterile or provides a sterile storage place for a brush head 15

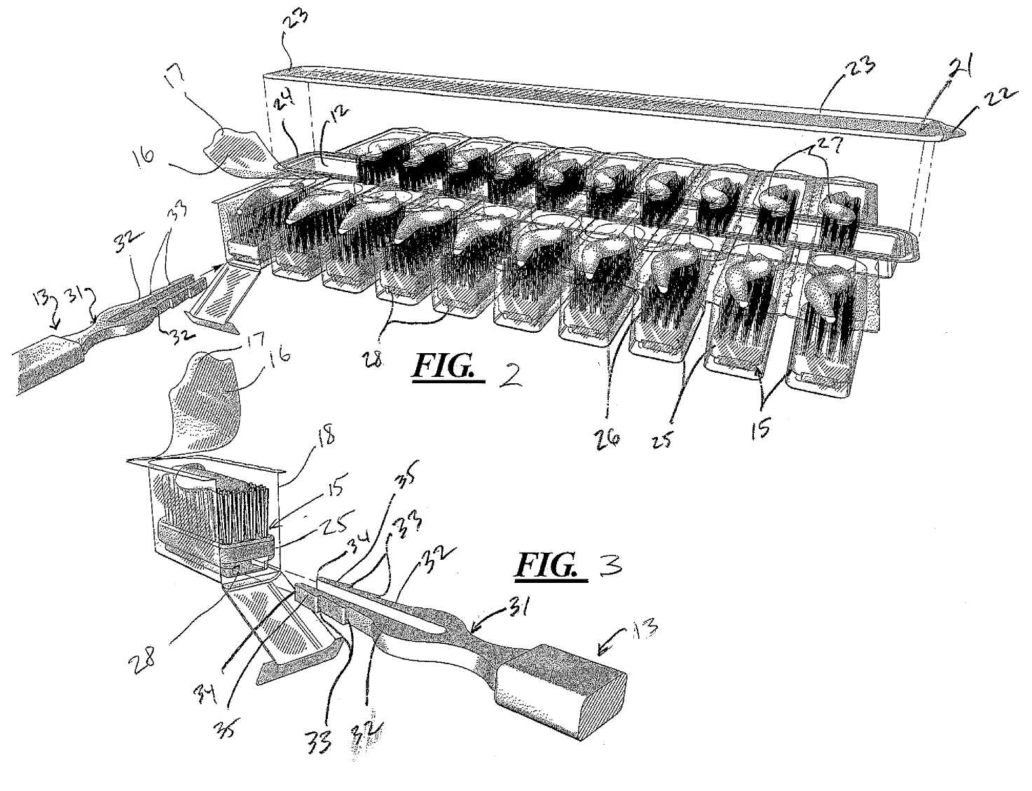

Similarly, the central trough 12 which accommodates the reusable handle 13 may also be equipped with a covet 21 that, in turn, is preferably equipped with a pull tab 22. The cover 21 may be glued or otherwise adhered to the trough 12 or, as shown in FIGS. 1 and 2, the outer periphery of the cover may appropriately shaped to mateably engage the corresponding outer periphery 24 of the trough 12

Each blush head 15 as shown in includes a head body 25 connected to a plurality of bristles or tufts of bristles shown generally at 26. The bristles 26 are coated with an appropriate amount of toothpaste 27. Pre-pasting the blush heads 15 avoids waste and the unwanted dripping of toothpaste on the sink or counter. The pre-pasting of the brush heads 15 is an excellent way to train young children good oral hygiene habits in a stepwise manner. Specifically, the child can easily connect the handle 13 to a blush head 15, brush his/her teeth and then dispose of the use brush. Manipulation of a toothpaste tube or requiring apparent to apply toothpaste to the blush head 15 is no longer necessary.

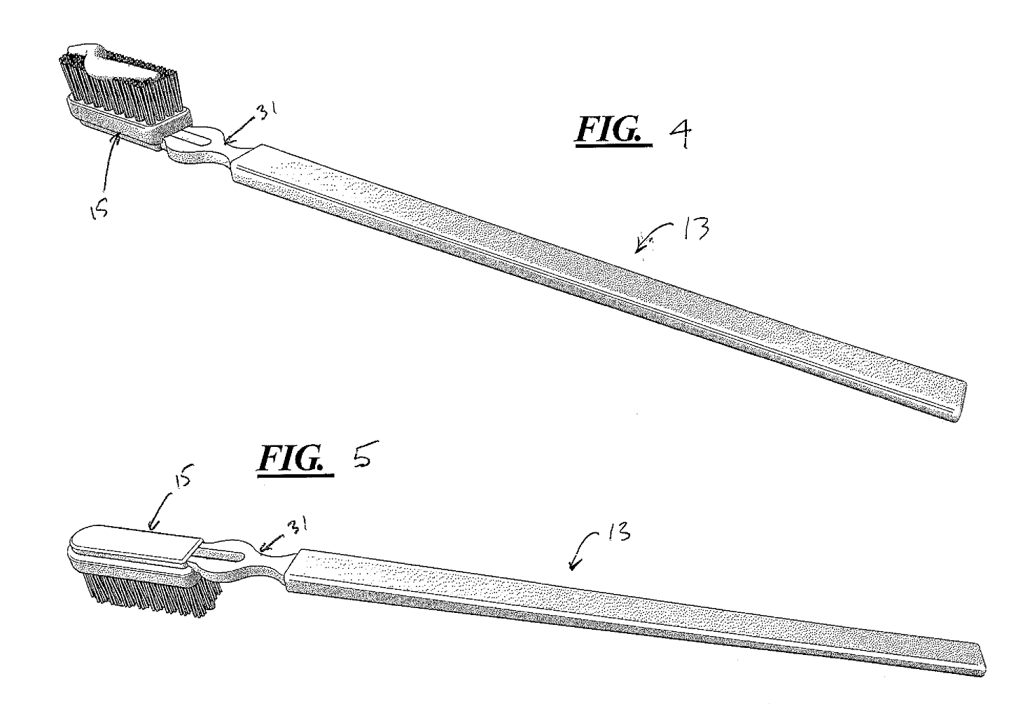

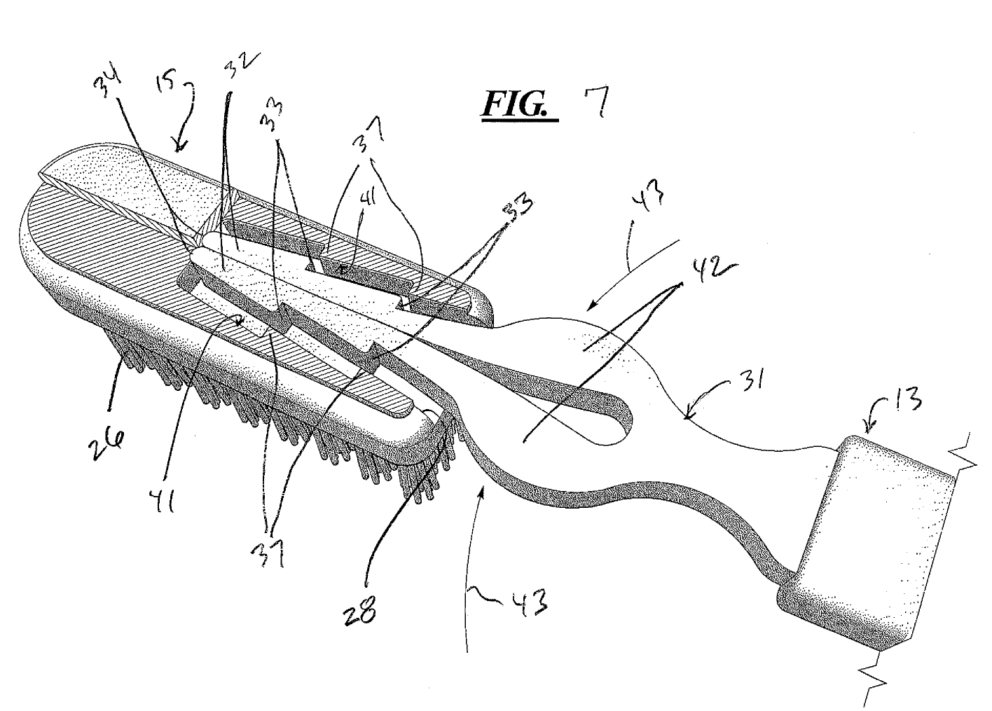

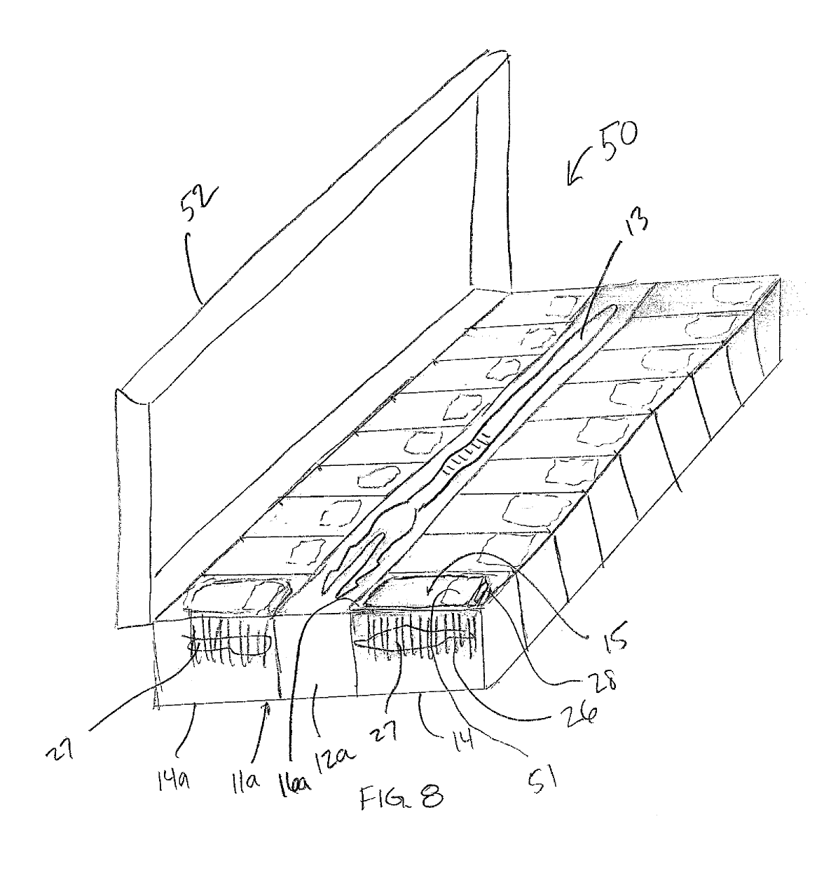

Turning to FIG. 3, the insertion of the handle 13 into a brush head 15 is extremely easy and can be performed safely by small children. Each brush head body 25 includes a shaped opening 28 the shaped opening 28 accommodates the shaped end 31 of the handle 13. More specifically, the shaped end 31 includes a pair of shaped prongs, both of which are shown at 32 in FIG. 3 and both of which are equipped with one or more barbs 33. The prongs 32 are essentially biased into position shown in FIGS. 2 and 3 and when the distal ends 34 enter the shaped opening 28, the prongs 32 are pressed inwardly toward each other as the ramped surfaces 35 engage the side walls of the shaped opening 28. As shown in FIGS. 6 and 7, the interior of the head 25 includes ridges 37 for engaging the barbs 33 and ramp surfaces 35 of the prongs 32

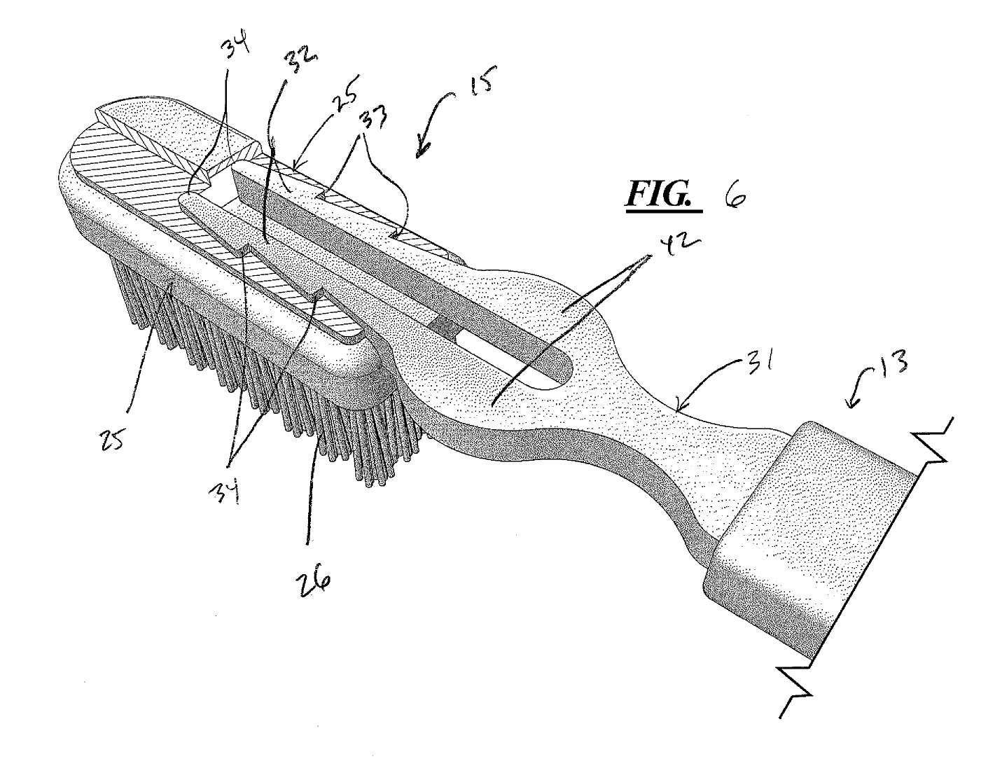

FIGS. 4 and 5 illustrate the handle 13 as detachably connected to a blush head 15. Simply put, a child or senior citizen will have no problems in inserting the dual-pronged and 31 of the handle 13 into the shaped opening 28 of a brush head 15. The handle may be inserted into the brush head with either side up. In other words, there is no upside down configuration for the handle with respect to the brush head. If a more ergonomic handle 13 is provided, then a single orientation with respect to the handle 13 and blush head 15 is preferred.

FIGS. 6 and 7 disclosed but one attachment and mechanism between a handle 13 and a brush head 15. The shaped opening 28 (see FIG. 7) includes a pair of opposing side walls 41 equipped with at least one ridge 37 for virtually engaging a barbed 33 disposed on one of the prongs 32 of the end 31 of the handle 13. To connect the handle 13 to the brush head 15, the end 31 of the handle 13 is simply inserted into the shaped opening 28. As shown in FIG. 7, to release the handle 13 from the brush at 15, the rounded transition sections 42 that connect the prongs 32 to the handle 13 are pressed inwardly in the direction of the arrows 43 as shown in FIG. 7. This action releases the barbs 33 from the ridges 37 and enables the end 31 of the handle 13 to be pulled out of the shaped opening 28 of the blush head 15.

A variety of other attachment mechanisms between the handle 13 and brush at 15 are available and will be apparent to those skilled in the art. A simple frictional engagement between the two elements may suffice. Further, other temporary locking mechanisms or threaded connections may suffice as well. This disclosure is simply not limited to the dual pronged handle and 31 illustrated herein or the dual barbed prongs 32 illustrated herein. Any temporary or releasable connection between the handle 13 and brush head 15 can be used. Preferably, these elements are manufactured using an injection molding process to keep the cost of manufacturing low.

It will be noted that the shape of the handle 13 may vary depending upon ergonomics preferences. This disclosure is not limited to the particular handle 13 shapes disclosed herein.

FIG. 8 discloses an alternative kit 50 that is equipped with a packaging 11a with side compartments 14a and a central trough for accommodating the handle 13. As opposed to the kit 10 described above in connection with FIGS. 1-3, the brush heads 15 are inverted with the bristles 26 facing downward in the shaped opening 28 disposed immediately beneath a perforated portion 51 of the cover 16a. The packaging 11a also includes a lid 52. Various other packaging styles and designs will be apparent to those skilled in the art.

While only certain embodiments have been set forth, alternatives and modifications will be apparent from the above description to those skilled in the art. These and other alternatives are considered equivalents and within the spirit and scope of this disclosure and the appended claims

What is claimed:

1. A dental care kit comprising:
a handle comprising an end comprising a shaped prong,
a plurality of heads, each head comprising a body connected to a plurality of bristles, at least some of the bristles being coated with toothpaste, each body of each head further comprising a shaped opening for frictionally and releasably receiving the shaped prong of the handle.

2. The kit of claim 1 wherein the end of the handle is flexible thereby permitting the prong to be moved into and out of functional engagement with one of the shaped openings of one of the heads.

3. The kit of claim 1 wherein the end of the handle comprises a pair of spaced-apart shaped prongs that can be frictionally and releasably received in one of the shaped openings of one of the heads.

4. The kit of claim 3 wherein the spaced-apart shaped prongs may be squeezed together to release the prongs from one of the shaped openings.

5. The kit of claim 1 wherein the shaped prong comprises at least one barb and each shaped opening comprises a sidewall with at least one ridge for frictionally engaging the barb of the prong.

6. The kit of claim 1 wherein the shaped prong comprises barbs and each shaped opening comprises a sidewall with ridges for frictionally engaging the barbs of the prong.

7. The kit of claim 3 wherein each shaped prong comprises at least one barb and each shaped opening comprises a pair of sidewalls each with at least one ridge for frictionally engaging the barb of the prong.

8. The kit of claim 3 wherein each shaped prong comprises barbs and each shaped opening comprises a pair of sidewalls each with ridges for frictionally engaging the barbs of the prong.

9. The kit of claim 3 wherein each prong is connected to the end of the handle by a rounded transition section, the rounded transition sections extending away from each other and comprising an outer surface that serves as a grip to facilitate the pressing of the prongs together.

10. The kit of claim 7 wherein each prong is connected to the end of the handle by a rounded transition section, the rounded transition sections extending away from each other and comprising an outer surface that serves as a grip to facilitate the pressing of the prongs together.

11. The kit of claim 8 wherein each prong is connected to the end of the handle by a rounded transition section, the rounded transition sections extending away from each other and comprising an outer surface that serves as a grip to facilitate the pressing of the prongs together.

12. The kit of claim 1 further comprising packaging wherein the packaging comprises a plurality of connected and sealed compartments with the handle being disposed within one of the sealed compartments and each head being within one of the other sealed compartments.

13. The kit of claim 12 wherein the compartments are sterile.

14. The kit of claim 13 wherein the kit comprises one handle and from about six to about 30 heads.

15. The kit of claim 13 wherein the kit comprises one handle and from about 10 to about 24 heads.

16. A dental hygiene kit comprising:
a packaging comprising a clear polymeric material and one large initially-sealed compartment for storing a handle and a plurality of smaller, initially-sealed compartments, each smaller compartment for storing one brush head, the compartments being connected to at least two other compartments, each compartment being sterile when sealed,
the handle comprising a fork and with two spaced-apart shaped prongs,
each head comprising a plurality of bristles connected to a body, at least some of the bristles being coated with toothpaste,
each body comprising a shaped hole with sidewalls for frictionally receiving the prongs of the handle.

17. The kit of claim 16 wherein each shaped hole comprises opposing sidewalls, each opposing side wall comprising at least one ridge for fictionally engaging one of the prongs and the prongs each comprise a barb for engaging one of the ridges.

18. The kit of claim 16 wherein the kit comprises from about six to about 24 heads.

19. A toothbrush with a pre-pasted removable head, the toothbrush comprising:
a handle comprising an elongated grip that terminates at a forked end with a pair of spaced-apart the barbed prongs, the prongs being connected to the grip by a flexible transition section that, when squeezed, causes the prongs to move inwardly towards each other,
a head comprising a body connected to a plurality of bristles, at least some of the bristles being pre-coated with toothpaste, the body comprising a shaped opening comprising sidewalls with ridges for frictionally engaging the barbed prongs.

20. The toothbrush of claim 19 wherein each prong is connected to an end of the handle by a rounded transition section, the rounded transition sections extending away from each other and comprising an outer surface that serves as a grip to facilitate the pressing of the transition sections and prongs together to release the prongs from the shaped opening of the head.
